(19) United States
(12) Patent Application Publication
(10) Pub. No.: US 2007/0077267 A1
(43) Pub. Date: Apr. 5, 2007

(54) Bioactive composite implants

(75) Inventors: Fred J. Molz IV, Collierville, TN (US); Hai H. Trieu, Cordova, TN (US)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(73) Assignee: SDGI Holdings, Inc.

(21) Appl. No.: 11/240,528

(22) Filed: Oct. 3, 2005

(51) Int. Cl. A61K 38/18; A61F 2/44; A61K 31/66

(52) U.S. Cl. 424/423; 623/17.11; 514/12; 514/102

(57) ABSTRACT

A composite spinal implant device including collagen and/or synthetic fibers impregnated with a bioactive formulation is disclosed. Also disclosed are methods of making the composite spinal implant devices, surgeries using the device, and kits containing the device.

### FIELD OF THE INVENTION

Embodiments relate to bioactive composite implants comprising one or more bioactive formulations impregnated into collagen and/or synthetic fibers. Cells and other nutrients also can be added to the bioactive composite prior to or during surgery.

### DESCRIPTION OF RELATED ART

Thousands of implant surgeries are performed every year in the United States on patients requiring biomedical implants. For example, more than 168,000 total hip replacements are performed each year in the United States alone. Shindle, M., et al., BioMechanics, 11(2):22-32 (2004).

Unfortunately, a number of implant surgeries each year require revision surgery to correct defects that have developed with the implant devices. For example, as discussed by Croci et al. regarding segmental resections of bone tumors, the increased rates of survival of patients having bone tumor resections has led to the discovery of the greater need for revision surgery of implant devices, where previously such observations were less frequent due to the unsuccessful oncologic management of the tumors. Croci et al., Rev. Hosp. Clin. Fac. Med. S. Paulo, 55(5):169-176 (2000).

Croci et al. state that the problems that have arisen related to the longer follow-up of endoprostheses implanted in bone tumor segmental resection patients include breaking and loosening of the implants, which are problems typically observed with total hip and knee replacements. Id at 170. Croci et al. further state that physicians conducting these intraoperative surgeries are familiar with the difficulties associated therewith, which include severe bone loss after removal of the implant and the cement.

Examples of problems associated with revision surgery are known. For example, once a bone stem is removed, the remaining revision site must be bored-out to remove the remaining materials and to provide a new surface for implantation of the replacement device(s). Often times, the remaining bone quality is poor, having a scalloped surface. Devices that do not require revision surgeries over time are desirable in order to avoid the problems associated with revision surgeries.

Devices designed to deliver osteoinductive agents in the vicinity of musculoskeletal implant devices are particularly useful in both primary and revision surgeries, in order to prevent the development of osteolysis in the vicinity of implant devices. Devices of this nature also are useful in preventing the need for future revision surgeries. Devices designed to deliver osteoinductive agents in the vicinity of musculoskeletal implants are particularly useful for both primary and revision surgeries involving total joint replacements, such as shoulder surgeries at the stem of the humeral component; in elbow surgeries, at the stem of the humeral and ulna components; in wrist surgeries, at the stem of the ulna component; in hip surgeries, at the femoral stem, associated with acetabular cup implants, and associated with bone screws; and in knee surgeries, at the femoral stem, at the back side of femoral component articulation, at the tibia stem, the underside of the tibia tray, and at the backside of the patella; and in total shoulder, total hip and total knee replacement surgeries.

In addition to the total joint replacement surgeries discussed previously, spine fusion surgery would benefit greatly from devices that contain or otherwise release bioactive agents, including bone growth promoting materials. Interbody fusion devices that include osteogenic materials are well known and described in, for example, U.S. Pat. Nos. 6,648,916 and 6,719,795, the disclosures of which are incorporated by reference herein in their entirety. Spinal fusion is indicated to provide stabilization of the spinal column for disorders such as structural deformity, traumatic instability, degenerative instability, and post resection iatrogenic instability. Fusion, or arthrodesis, can thus be achieved, for example, by the formation of an osseous bridge between adjacent motion segments.

The fusion can be accomplished either anteriorly between contiguous vertebral bodies or posteriorly between consecutive transverse processes, laminae or other posterior aspects of the vertebrae. Typically, the osseous bridge, or fusion mass, is biologically produced by recreating conditions of skeletal injury along a “fusion site” and allowing the normal bone healing response to occur. This biologic environment at a proposed fusion site requires the presence of osteogenic or osteopotential cells, adequate blood supply, sufficient inflammatory response, and appropriate preparation of local bone. To this end, a process known as decortication is typically used-to prepare bone and increase the likelihood of fusion. Decortication involves removing the outer cortex of spinal bone with a burr to induce bleeding bone and release bone marrow. Decortication also initiates the inflammatory response, releases osteoinductive cytokines, provides additional osteogenic cells, and creates a host attachment site for the subsequent fusion mass. Bone graft materials are often used to promote spinal fusions. Autogenous iliac crest cortico-cancellous bone is presently a widely-used bone grafting material.

In early spinal fusion techniques, bone material, or bone osteogenic fusion devices, were simply positioned between adjacent vertebrae, typically at the posterior aspect of the vertebrae. In the early history of these osteogenic fusion devices, the osteogenic fusion devices were formed of cortical-cancellous bone. Consequently, the spine was stabilized by way of screws, plates and/or rods spanning the affected vertebrae. With this technique, once fusion occurred across and incorporating the bone osteogenic fusion device, the hardware used to maintain the stability of the spine became superfluous.

Following the successes of the early fusion techniques, focus was directed to modifying the device placed within the intervertebral space to support and fuse together adjacent vertebrae by posterior-fusion or anterior grafting. For example, surgical prosthetic implants for vertebrae described in U.S. Pat. No. 5,827,328 include rigid annular plugs that have ridged faces to engage adjacent vertebrae to resist displacement and allow ingrowth of blood capillaries and packing of bone graft. These annular implants are usually made of biocompatible carbon fiber reinforced polymers, or traditional orthopaedic implant materials such as nickel, chromium, cobalt, stainless steel or titanium. The individual implants are internally grooved and are stacked against each other to form a unit between the two adjacent vertebrae.

Another intervertebral fusion device described in U.S. Pat. No. 5,397,364, which includes an assembly of two lateral spacers and two central spacers, which defines a channel in the center of the fusion device for insertion of bone graft material. The spacers are maintained in their configuration within the intradiscal space by screws threaded into a vertebra from the outside of the disc.

Cylindrical hollow implants or “cages” are represented by the patents to Bagby, U.S. Pat. No. 4,501,269; Brantigan, U.S. Pat. No. 4,878,915; Ray, U.S. Pat. No. 4,961,740; and Michelson, U.S. Pat. No. 5,015,247, the disclosures of each of which are incorporated by reference herein in their entirety. The outer wall of the cage creates an interior space within the cylindrical implant that is filled with bone chips, for example, or other bone growth-inducing material such as hydroxyapatite or BMP. The cylindrical implant can include a threaded exterior to permit threaded insertion into a tapped bore formed in the adjacent vertebrae. One fusion cage implant is disclosed in U.S. Pat. No. 5,026,373 to Ray et al. The Ray '373 fusion cage includes apertures extending through its wall which communicate with an internal cavity of the cage body. The adjacent vertebral bone structures communicate through the apertures with bone growth inducing substances within the internal cavity to unite and eventually form a solid fusion of the adjacent vertebrae. Other prosthetic implants are disclosed in U.S. Pat. Nos. 4,501,269, 4,961,740, 5,015,247 and 5,489,307, the disclosures of which are incorporated by reference herein in their entirety. Other fusion implants have been designed to be impacted into the intradiscal space.

Experience over the last several years with these interbody fusion devices has demonstrated the efficacy of these implants in yielding a solid fusion. Variations in the design of the implants have accounted for improvements in stabilizing the motion segment while fusion occurs. Nevertheless, some of the interbody fusion devices still have difficulty in achieving a complete fusion, at least without the aid of some additional stabilizing device, such as a rod or plate. Moreover, some of the devices are not structurally strong enough to support the heavy loads and bending moments applied at certain levels of the spine, namely those in the lumbar spine. In addition, some of the devices become contaminated, or by virtue of their extra-body construction, evoke an adverse immune response when implanted.

Even with devices that do not have these difficulties, other less desirable characteristics exist. Recent studies have suggested that the interbody fusion implant devices, or cages as they are frequently called, lead to stress-shielding of the bone within the cage. It is well known that bone growth is enhanced by stressing or loading the bone material. The stress-shielding phenomenon relieves some or all of the load applied to the material to be fused, which can greatly increase the time for complete bone growth, or disturb the quality and density of the ultimately formed fusion mass. In some instances, stress-shielding can cause the bone chips or fusion mass contained within the fusion cage to resorb or evolve into fibrous tissue rather than into a bony fusion mass.

A further difficulty encountered with many fusion implants is that the material of the implant is not radiolucent. Most fusion cages are formed of metal, such as stainless steel, titanium or porous tantalum. The metal of the cage shows up prominently in any radiograph (x-ray) or CT scan. Since most fusion devices completely surround and contain the bone graft material housed within the cage, the developing fusion mass within the metal cage between the adjacent vertebrae cannot be seen under traditional radiographic visualizing techniques and only with the presence of image scatter with CT scans. Thus, the spinal surgeon does not have a means to determine the progress of the fusion, and in some cases cannot ascertain whether the fusion was complete and successful.

Various bone grafts and bone graft substitutes have been used to promote osteogenesis and to avoid the disadvantages of metal implants, such as stress shielding and radiographic issues. Autograft is often preferred because it is osteoinductive. Both allograft and autograft are biological materials that are replaced over time with the patient's own bone, via the process of creeping substitution. Over time, a bone graft virtually disappears unlike a metal implant, which persists long after its useful life.

It is believed that the use of bone grafts avoids stress shielding because bone grafts have a similar modulus of elasticity as the surrounding bone. Commonly used implant metallic materials have stiffness values far in excess of both cortical and cancellous bone. Titanium alloy has a stiffness value of 114 Gpa and 316L stainless steel has a stiffness of 193 Gpa. Cortical bone, on the other hand, has a stiffness value of about 17 Gpa. Moreover, bone as an implant also allows excellent postoperative imaging because it does not cause scattering like metallic implants on CT or MRI imaging.

Various implants have been constructed from bone or graft substitute materials to fill the intervertebral space after the removal of the disc. For example, the Cloward dowel is a circular graft made by drilling an allogeneic or autogeneic plug from the illium. Cloward dowels are bicortical, having porous cancellous bone between two cortical surfaces. Such dowels have relatively poor biomechanical properties, in particular a low compressive strength. Therefore, the Cloward dowel is not suitable as an intervertebral spacer without internal fixation due to the risk of collapsing prior to fusion under the intense cyclic loads of the spine.

Bone dowels having greater biomechanical properties have been produced and marketed by the University of Florida Tissue Bank, Inc., 1 Progress Boulevard, P.O. Box 31, S. Wing, Alachua, Fla. 32615. Unicortical dowels from allogeneic femoral or tibial condyles are available. The University of Florida has also developed a diaphysial cortical dowel having superior mechanical properties. This dowel also provides the further advantage of having a naturally preformed cavity formed by the existing meduallary canal of the donor long bone. The cavity can be packed with osteogenic materials such as bone or bioceramic.

Unfortunately, the use of bone grafts presents several disadvantages. Autograft is available in only limited quantities. The additional surgery also increases the risk of infection and blood loss and may reduce structural integrity at the donor site. Furthermore, some patients complain that the graft harvesting surgery causes more short-term and long-term pain than the fusion surgery. Allograft material, which is obtained from donors of the same species, is more readily obtained. However, allogeneic bone does not have the osteoinductive potential of autogenous bone and therefore may provide only temporary support. The slow rate of fusion using allografted bone can lead to collapse of the disc space before fusion is accomplished.

Both allograft and autograft present additional difficulties. Graft alone may not provide the stability required to withstand spinal loads. Internal fixation can address this problem but presents its own disadvantages such as the need for more complex surgery as well as the disadvantages of metal fixation devices. In addition, the surgeon often is required to repeatedly trim the graft material to obtain the correct size to fill and stabilize the disc space. This trial and error approach increases the length of time required for surgery. Furthermore, the graft material usually has a smooth surface that does not provide a good friction fit between the adjacent vertebrae. Slippage of the graft may cause neural and vascular injury, as well as collapse of the disc space. Even where slippage does not occur, micromotion at the graft/fusion-site interface may disrupt the healing process that is required for fusion.

Several attempts have been made to develop a bone graft substitute that avoids the disadvantages of metal implants and bone grafts, while capturing advantages of both. For example Unilab, Inc. markets various spinal implants composed of hydroxyapatite and bovine collagen. In each case developing an implant having the biomechanical properties of metal and the biological properties of bone without the disadvantages of either has been extremely difficult or impossible to achieve.

These disadvantages have led to the investigation of bioactive substances that regulate the complex cascade of cellular events of bone repair. Such substances include bone morphogenetic proteins, for use as alternative or adjunctive graft materials. Bone morphogenetic proteins (BMPs), a class of osteoinductive factors from bone matrix, are capable of inducing bone formation when implanted in a fracture or surgical bone site. Recombinantly produced human bone morphogenetic protein-2 (rhBMP-2) has been demonstrated in several animal models to be effective in regenerating bone in skeletal defects. The use of such proteins has led to a need for appropriate carriers and fusion spacer designs, when used in spinal fusion surgery.

Due to the need for safer bone graft materials, bone graft substitutes, such as bioceramics, have recently received considerable attention. The challenge has been to develop a bone graft substitute that avoids the disadvantages of metal implants and bone grafts while capturing the advantages of both. Calcium phosphate ceramics are biocompatible and do not present the infectious or immunological concerns of allograft materials. Ceramics may be prepared in any quantity, which is a great advantage over autograft bone graft material. Furthermore, bioceramics are osteoconductive, stimulating osteogenesis in boney sites. Bioceramics provide a porous matrix which further encourages new bone growth. Unfortunately, ceramic implants typically lack the strength to support high spinal loads and therefore require separate fixation before the fusion.

Hydroxyapatite (HA) and tricalcium phosphate ceramics are the most commonly used calcium phosphate (TCP) ceramics for bone grafting. Hydroxyapatite is chemically similar to inorganic bone substance and biocompatible with bone. However, it is slowly degraded. β-tricalcium phosphate is rapidly degraded in vivo and is too weak to provide support under the cyclic loads of the spine until fusion occurs. Again, it has been difficult to develop a spinal implant that has strength characteristics similar to the metal, ceramic, or metal alloy implants, but that also has osseointegration characteristics similar to bone.

The description herein of disadvantages and deleterious properties associated with known apparatus, methods, compositions, and devices is not intended to limit the scope of the invention to their exclusion. Indeed, various embodiments of the invention may include one or more known apparatus, methods, compositions, and devices without suffering from the disadvantages and deleterious properties described herein.

### SUMMARY OF THE EMBODIMENTS

There remains a need for orthopaedic implant devices that continue to maintain a high level of strength and utility over time. There also remains a need to develop orthopaedic implant devices that can be fabricated from a wide variety of materials or composites other than metal, that can promote bone growth and fusion, and that preferably do not elicit adverse immune responses when implanted.

Applicants describe herein preferred spinal implant devices that fulfill the remaining need in the art for implant devices that continue to function while resisting the effects of osteolysis, bone loss, weakening over time, that can be fabricated from a wide variety of materials or composites other than just metal, that can promote bone growth, and that do not elicit adverse immune responses when implanted.

Features of embodiments of the invention therefor provide a composite spinal implant useful for promoting in-growth of bone and vascular tissue. The implant of embodiments includes a spinal implant device comprised of a composite material that includes at least collagen and/or a synthetic fiber, whereby the collagen and/or synthetic fiber is impregnated with a bioactive formulation capable of promoting bone growth between bone and the implant device. The bioactive formulation may be present on or at the surface of the implant device, or may be impregnated in the device below the surface.

Features of an additional embodiment provide a method of making a composite spinal implant that includes providing a composite implant composition comprising at least collagen and/or synthetic fibers, forming the composite implant composition into a spinal implant, and impregnating the collagen and/or synthetic fibers with a bioactive formulation. Impregnation may take place prior to, during, or after forming the composite implant composition into a spinal implant.

Additional embodiments include methods of performing spinal surgery using the composite spinal implants, as well as kits containing the composite spinal implants. These and other features of the invention will be readily apparent to those skilled in the art upon reading the detailed description that follows.

### DETAILED DESCRIPTION OF PREFERRED EMBODIMENTS

For the purposes of promoting an understanding of the embodiments described herein, reference will now be made to preferred embodiments and specific language will be used to describe the same. The terminology used herein is for the purpose of describing particular embodiments only, and is not intended to limit the scope of the present invention. As used throughout this disclosure, the singular forms “a,” “an,” and “the” include plural reference unless the context clearly dictates otherwise. Thus, for example, a reference to “an implant” includes a plurality of such implants, as well as a single implant, and a reference to “an osteoinductive agent” is a reference to one or more agents and equivalents thereof known to those skilled in the art, and so forth.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications mentioned herein are cited for the purpose of describing and disclosing the various implants, osteoinductive agents, and other components that are reported in the publications and that might be used in connection with the invention. Nothing herein is to be construed as an admission that the invention is not entitled to antedate such disclosures by virtue of prior invention.

As used herein, “orthopaedic device” shall mean any bone implant including, but not limited to, endoprostheses and other devices designed to replace or supplement endogenous bone structures in the body. “Orthopaedic device” further encompasses dental devices such as replacement teeth and other dental implants. The expression “spinal implant” refers to any device intended to be implanted into the body that serves to support the spine or assist in correcting a spinal deformity.

As used herein, “bioavailable” shall mean that the isolated osteoinductive agents(s) are provided in vivo in the patient, wherein the isolated osteoinductive agent(s) retain biological activity. By retaining biological activity is meant that the isolated osteoinductive agent(s) retain at least 25% activity, more preferably at least 50% activity, still more preferably at least 75% activity, and most preferably at least 95% or more activity of the isolated osteoinductive agent relative to the activity of the isolated osteoinductive agent prior to implantation.

As used herein, “mature polypeptide” shall mean a post-translationally processed form of a polypeptide. For example, mature polypeptides may lack one or more of a signal peptide and a propeptide domain following expression in a host expression system.

As used herein, “immediate release” shall mean formulations of the invention that provide the osteoinductive formulations in a reasonably immediate period of time.

As used herein, “sustained release” shall mean formulations of the invention that are designed to provide osteoinductive formulations at relatively consistent concentrations in bioavailable form over extended periods of time.

As used herein, “isolated” shall mean material removed from its original environment (e.g., the natural environment if it is naturally occurring), and thus is altered “by the hand of man” from its natural state. For example, an isolated polynucleotide could be part of a vector or a composition of matter, or could be contained within a cell, and still be “isolated” because that vector, composition of matter, or particular cell is not the original environment of the polynucleotide.

The expression “synthetic fiber” as used herein denotes any fiber that is not a natural fiber, but rather is a fiber made by manipulation or modification of a natural fiber, or a fiber synthesized from polymers or other chemical entities. Synthetic fiber also denotes these synthetic fibers capable of being molded into an implant shape, and capable of being impregnated with one or more of the bioactive formulations described herein.

Embodiments of the invention relate to bioactive composite implants, preferably orthopaedic implants, and most preferably spinal implants, that contain a bioactive formulation dispersed within the implant, or dispersed within at least a portion f the surface of the composite implant. It is preferred that the bioactive formulation be useful for promoting the in-growth of bone, cartilage, or related tissues from neighboring tissues at the site of implantation of the composite implant. The composite implants optionally provides the bioactive formulations as an immediate release or a sustained-release formulation useful for promoting sustained in-growth of endogenous bone in the patient.

Composite implant devices useful in the embodiments include, but are not limited to, orthopaedic devices having a surface capable of releasing at least a portion of the bioactive formulation, while providing adequate structural support to the patient in the implant location. Non-limiting examples of composite implant devices include, but are not limited to, implants created from ceramic or metals that are then coated or admixed with with a collagen or synthetic fiber material impregnated with the bioactive formulation, or implants that are fabricated from the collagen or synthetic fiber material. Skilled artisans are capable of fabricating a suitable implant device with a composite of ceramic and/or metal, together with collagen or a synthetic fiber, (or the collagen or synthetic fiber alone) whereby the collagen or synthetic fiber either forms a part of the implant, or merely serves as a coating on at least a portion of the surface of the implant. To aid in osseointegration, the surface of the composite implant may be roughened, or made porous. General methods of manufacturing spinal implant devices with porous or roughened surfaces are well known in the art and include, for example the use of sintering beads, machining of device surfaces, laser etching of surfaces, using nanotube technology to create roughened surfaces, casting roughened surfaces, and chemically or mechanically etching or machining roughened surfaces.

In one embodiment, the composite material comprises a composite of metal and collagen or synthetic fiber materials that are molded together to form the implant. In another embodiment, the composite material includes a composite of ceramic and collagen or synthetic fiber materials that are formed together to form the implant. Another embodiment includes a composite implant prepared by collagen alone, synthetic fibers alone, or a mixture of collagen and synthetic fibers. These composite implants may be molded or formed using any suitable molding or forming technique, including, injection molding, pressing (e.g., hot isostatic pressing), extrusion molding, cast molding, formation of green ceramic tapes and subsequent firing and then impregnation with the bioactive formulation, sintering, and the like. Those skilled in the art recognize other suitable molding or forming methodologies useful in forming the composite implants described herein.

If the composite implant includes a porous or roughened surface to facilitate the impregnation of a bioactive formulation coating, the outer coating may be deposited on the implant substrate after synthesis of the implant device, or substantially concurrent with or prior to the synthesis of the implant device substrate. In another embodiment, the composite implant device substrate itself harbors a porous surface that functions to provide the porous surface into which the bioactive formulations may be applied.

In one preferred embodiment of the invention, the composite implant device(s) are modified to include a porous substrate similar to that described in U.S. Pat. No. 5,282,861 to Kaplan, the entire disclosure of which is herein incorporated by reference. The composite implant device(s) may include a composite material having a reticulated open cell carbon foam substrated infiltrated with tantalum or niobium, or alloys thereof, by the chemical vapor deposition (CVD) process. Other metals such as niobium, hafnium and/or tungsten could be alloyed with the tantalum or hafnium and/or tungsten with niobium to change modulus and/or strength of the implant device.

The carbon foam can be infiltrated by chemical vapor deposition (CVD). The resulting lightweight, strong, porous structure, mimicking the microstructure of natural cancellous bone, acts as a matrix for the incorporation of bone and for the reception of and impregnation of the bioactive formulation or cells and tissue. The pores of the matrix preferably are connected to one another to form continuous, uniform channels with no dead ends. This intricate network of interconnected pores provides optimal permeability and a high surface area to encourage cell and tissue in-growth, vascularization, and deposition of new bone.

The result is a new composite bioactive implant that, when placed next to bone or tissue, initially serves as a prosthesis and then functions as a scaffold for regeneration of normal tissues. The porous nature of the resulting implant material is particularly well suited for impregnation with bioactive formulations. The implant offers the potential for use in alveolar ridge augmentation, periodontics, and orthognathic reconstruction, and is even more particularly suited for use in spinal implant devices where regeneration of tissues and/or bone are highly desirable. The composite implant described in the embodiments herein also is superior to known spinal implants that utilize carriers (e.g., collagen or synthetic fibers) soaked with BMP, etc., and that are positioned within or surrounding a metallic or ceramic implant. The known spinal implants can easily be separated from the carriers or the carriers may be too readily resorbed into the body to provide the requisite osseointegration. In contrast, the composite implants of the preferred embodiments are actually made from the collagen and/or synthetic fibers, and consequently, do not suffer from some of the disadvantages associated with the known systems.

The composite implant devices according to the embodiments described herein preferably include collagen and/or synthetic fibers that are impregnated with bioactive formulations. The collagen and/or synthetic fibers may be impregnated with the bioactive formulation prior to, during, or after formation of the implant, and preferably, they are impregnated after formation of the implant. Impregnation after implant formation reduces the loss of bioactive formulation and activity that may occur during implant formation, especially when high pressures and/or temperatures are involved in the implant formation procedure.

Fabricating a composite implant with collagen and/or synthetic fiber may provide an implant device with less structural integrity or strength initially, when compared to rigid metallic or ceramic implants, but that quickly surpasses the structural integrity of conventional metallic or ceramic devices due to the enhanced osseointegration. For example, if used as a spinal fusion cage, the composite implant devices of the embodiments preferably are designed to absorb less vertebral body load, and optionally flex in response to the load, thereby placing more stress on the bioactive formulation impregnated therein, and in turn inducing greater osseointegration. It is preferred that the composite spinal implants have a stiffness less than stainless steel and titanium, and preferably have a stiffness roughly similar to the stiffness of cortical bone. The composite spinal implants of the embodiments therefore may have a stiffness within the range of from about 10 Gpa to about 50 Gpa, more preferably within the range of from about 12 Gpa to about 25 Gpa, and most preferably within the range of from about 15 Gpa to about 20 Gpa. Methods of fabricating orthopedic and spinal implants with a material containing synthetic fibers and/or collagen are disclosed in, for example, U.S. Pat. Nos. 6,719,795; 6,607,530; 6,648,916; 6,423,095; 6,371,988; 6,261,586; 6,221,109; 6,039,762; 6,008,433; 5,885,292; 5,741,261, and 5,348,026, the disclosures of each of which are incorporated by reference herein in their entirety.

The collagen and/or synthetic fiber used in the embodiments can be any biologically acceptable component capable of being impregnated and retaining at least initially, the bioactive formulations described herein. The collagen and/or synthetic fiber therefore can be considered a carrier for the bioactive formulation. The bioactive formulation may contain, however, an additional carrier as a carrier for the bioactive agents (e.g, osteoconductive and/or osteoinductive agents), that may be the same as or similar to the collagen or synthetic fiber. The carrier may be any suitable medium capable of delivering the bioactive formulation to the surrounding tissue. Such carriers are well known and commercially available.

Any collagen may be used in the embodiments so long as it is biocompatible, capable of being impregnated with the bioactive formulation, and capable of being formed into a spinal implant device. Examples of suitable collagen include, but are not limited to, human collagen type I, human collagen type II, human collagen type III, human collagen type IV, human collagen type V, human collagen type VI, human collagen type VII, human collagen type VIII, human collagen type IX, human collagen type X, human collagen type XI, human collagen type XII, human collagen type XIII, human collagen type XIV, human collagen type XV, human collagen type XVI, human collagen type XVII, human collagen type XVIII, human collagen type XIX, human collagen type XXI, human collagen type XXII, human collagen type XXIII, human collagen type XXIV, human collagen type XXV, human collagen type XXVI, human collagen type XXVII, and human collagen type XXVIII, and combinations thereof. Collagen further may comprise, or alternatively consist of, hetero- and homo-trimers of any of the above-recited collagen types. In a preferred embodiment, the collagen comprises, or alternatively consist of, hetero- or homo-trimers of human collagen type I, human collagen type II, and human collagen type III, or combinations thereof.

The collagen may be human or non-human, as well as recombinant or non-recombinant. In a preferred embodiment, the collagen is recombinant collagen. Methods of making recombinant collagen are known in the art, for example, by using recombinant methods such as those methods described in U.S. Pat. Nos. 5,895,833 (trangenic production), J. Myllyharju, et al., Biotechnology of Extracellular Matrix, 353-357 (2000) (production of recombinant human types I-III in Pichia pastoris), Wong Po Foo, C., et al., Adv. Drug Del. Rev., 54:1131-1143 (2002), or by Toman, P. D., et al., J. Biol. Chem., 275(30):23303-23309 (2001), the disclosures of each of which are herein incorporated by reference. Alternatively, recombinant human collagen types may be obtained from commercially available sources, such as for example, as provided by FibroGen (San Francisco, Calif.).

One preferred collagen is an absorbable collagen sponge marketed by Integra LifeSciences Corporation under the trade name HELISTAT® Absorbable Collagen Hemostatic Agent. Other suitable materials are BIOGIDE®, BIO-OSS®, and BIO-OSS COLLAGEN®, all commercially available from Ed. Geistlich Sohne AG fur Chemische Industrie, Switzerland, as described in U.S. Pat. Nos. 5,167,961, 5,417,975, 5,573,771, and 5,837,278, the disclosures of each of which are incorporated by reference herein in their entirety.

Suitable synthetic fibers for use in the embodiments are any biocompatible fibers that can be impregnated with a bioactive formulation, and that can be shaped into a suitable implant and have the requisite strength characteristics. Any of the known synthetic fibers suitable for forming a biocompatible implant can be used in the embodiments, so long as the fibers also are capable of being impregnated with a bioactive formulation. Without intending on being bound by any theory of operation, the inventors believe that impregnating the collagen and/or synthetic fibers with the bioactive formulation provides superior osseointegration with adjacent bone, when compared to merely coating the materials with a bioactive formulation because impregnation provides a more uniform and secure junction between the materials.

It also is preferred that the synthetic fibers used in the embodiments be absorbable. In surgery it is known to use implants, or their parts or components, which are manufactured at least partially of an absorbable polymer and/or of a polymer composite containing reinforcing elements, for fixation of bone fractures, osteotomies or arthrodeses, joint damages, tendon and ligament damages etc. Such implants include e.g. rods, screws, plates, intramedullary nails and clamps, all of which are useful implants herein.

U.S. Pat. Nos. 3,620,218 and 3,739,733 describe rods, screws, plates, and cylinders manufactured from polyglycolic acid. U.S. Pat. No. 4,052,988 describes absorbable sutures and other surgical devices manufactured of polydioxanone. U.S. Pat. No. 4,279,249 describes osteosynthesis devices that are manufactured of polylactide or of copolymer containing a plurality of of lactide units, which matrix has been reinforced with reinforcing elements manufactured of polyglycolide or of copolymer including mainly glycolic acid units. The disclosures of each of these patents are incorporated by reference herein in their entireties.

DE 2947985 A1 describes at least partially degradable composites that comprise a copolymer of methylmethacrylate and N-vinlpyrrolidone, again reinforced with polyamide fibers or with oxycellulose fibers. U.S. Pat. No. 4,243,775 describes surgical products manufactured of copolymer of glycolic acid and trimethylene carbonate. U.S. Pat. No. 4,329,743, describes a composite of a bio-absorbable polymer and carbon fibers, which composite is suitable for manufacturing surgical articles. U.S. Pat. No. 4,343,931 describes absorbable polyesteramides, which are suitable for manufacturing of surgical implants. The disclosures of each of these United States patents are incorporated by reference herein in their entireties.

European Patent Application EPO 0,146,398 describes a method for manufacturing of biodegradable prosthesis about a biodegradable polymer matrix that is reinforced with biodegradable ceramic fibers. WO 86/00533 describes an implant material for reconstructive surgery of bone tissue, which material comprises a biodegradable porous polymer material and biodegradable or biostable fibers. D. Tunc, A High Strength Absorbable Polymer for Internal Bone Fixation, 9th Annual Meeting of the Society for Biomaterials, Birmingham, Ala., Apr. 27-May 1, 1983, p. 17, describes a high strength absorbable polylactide, with an initial tensile strength about 50-60 MPa and which material retains a significant part of its initial strength 8-12 weeks after the implantation. This material can be considered suitable to be applied as a basic material in manufacturing of internal bone fixation devices that are totally absorbable in living tissues. D. Tunc, et al., Evaluation of Body Absorbable Bone Fixation Devices, 31st Annual ORS, Las Vegas, Nev., Jan. 21-24, 1985, p. 165, describes high strength, totally absorbable polylactide (initial strength 57,1 MPa), which was used as plates and screws for fixation of canine radial osteotomies. D. Tunc, et al., Evaluation of Body Absorbable Screw in Avulsion Type Fractures, the 12th Annual Meeting of the Society for Biomaterials, Minneapolis-St. Paul, Minn., USA, May 29 to Jun. 1, 1986, p. 168, describes the application of high strength polylactide screws in fixation of avulsion-type fractures (fixation of canine calcaneus osteotomy).

U.S. Pat. No. 4,776,329 describes a compression screw comprising a non-absorbable compression parts and a screw. At least the head of the screw comprises material, which is resorbable in contact with tissue fluids. Self-reinforced absorbable fixation devices have significantly higher strength values than the non-reinforced absorbable fixation devices. U.S. Pat. No. 4,743,257 describes a self-reinforced surgical composite material, which comprises an absorbable polymer or copolymer, which has been reinforced with absorbable reinforcing elements, which have the same chemical element composition as the matrix. U.S. Pat. No. 5,348,026 discloses an osteoconductive bone screw comprised of a plurality of synthetic pre-torqued fibers coated with an osteoconductive material such as BMP. The disclosures of these United States patents also are incorporated by reference herein in their entirety.

The following patents relate to absorbable (biodegradable or resorbable) polymers, copolymers, polymer mixtures, or composites: U.S. Pat. No. 3,297,033; U.S. Pat. No. 3,636,956, U.S. Pat. No. 4,052,988; U.S. Pat. No. 4,343,931; U.S. Pat. No. 3,969,152; U.S. Pat. No. 4,243,775; FI Patent Appln. No. 85 5079, Fl Pat. Appln. No. 86 0366; FI Patent Appln. No. 86 0440 and FI Pat. Appln. No. 88 5164. The disclosures of each of these United States patents are incorporated by reference herein in their entireties.

Preferred synthetic fibers for use in the embodiments therefore include any of the afore-mentioned synthetic fibers. In the present embodiments, however, the fibers are impregnated with a bioactive formulation, and do not necessarily require (although in one embodiment they may include) additional reinforcing materials. The synthetic fiber materials include polyglycolic acid, polydioxanone, polyglycolide, a copolymer containing glycolicacid units, a copolymer of methylmethacrylate and N-vinylpyyrolidone, polyamide, oxycellulose, copolymer of glycolic acid and trimethylene carbonate, polyesteramides, polylactide, polyetheretherketone, polymethylmethacrylate, fibrillated absorbable materials, and mixtures and combinations thereof.

The composite spinal implants of the embodiments may include any known spinal implant or later discovered spinal implant. Suitable spinal implants include, for example, fusion cages, (lumbar and cervical), cervical and lumbar plates, rods, screws, hooks, anchors, fasteners, ligaments, nucleus replacement devices, intramedullary nails, clamps, facet arthroplasty devices, and the like.

The collagen and/or synthetic fibers utilized in accordance with the embodiments described herein are impregnated with a bioactive formulation. It is preferred to impregnate the collagen and/or synthetic fibers with the bioactive formulation by coating the collagen and/or synthetic fibers with the bioactive formulation. After impregnation, the collagen and/or synthetic fibers by themselves, or together with metal, a metal alloy, or a ceramic material can be combined and then molded to form the spinal implant. Alternatively, the collagen and/or synthetic fiber can be formed into a spinal implant and then contacted with a bioactive composition to impregnate the collagen or synthetic fiber. To facilitate impregnation, the formed implant can be subjected to additional treatments such as roughening of the surface, grinding, polishing, etching, mechanical surfacing, growth of nanotubes, etc. Using the guidelines provided herein, a skilled artisan will appreciate the myriad methodologies suitable to impregnate the collagen and/or synthetic fiber with a bioactive formulation, depending on the type of implant, the structure and chemical make-up of the implant, etc.

The bioactive formulations that can be used in the embodiments described herein include one or more osteoinductive agents, and/or osteoconductive agents, and provide the one or more agents in bioavailable form in immediate release or sustained release formulations. Bioactive formulations further optionally comprise one or more of the following components: antibiotics, carriers, bone marrow aspirate, bone marrow concentrate, demineralized bone matrix, immunosuppressives, agents that enhance isotonicity and chemical stability, and any combination of one or more, including all, of the recited components.

The obioactive formulations of the invention are available as immediate release formulations or sustained release formulations. One of skill in the art of implant surgery is able to determine whether a patient would benefit from immediate release formulations or sustained release formulations based on factors such as age and activity level. Therefore, the bioactive formulations of the embodiments are available in immediate or sustained release formulations.

Representative immediate release formulations are liquid formulations comprising at least osteoinductive agent(s) that are impregnated into the composite implant, and remain available in liquid form in vivo. The liquid formulations provide the osteoinductive agent in bioavailable form at rates that are dictated by the fluid properties of the liquid formulation, such as diffusion rates at the site of implantation, the influence of endogenous fluids, etc. Examples of suitable liquid formulations comprise water, saline, or other acceptable fluid mediums that will not induce host immune responses.

Immediate release formulations provide the bioactive formulation in a reasonably immediate period of time, although factors such as proximity to bodily fluids, density of application of the formulations, etc, will influence the period of time within which the bioactive agent is liberated from the formulation. However, immediate release formulations are not designed to retain the one or more bioactive agents for extended periods of time, and typically will lack a biodegradable polymer.

In another embodiment, bioactive formulations are available in sustained release formulations that provide the osteoinductive agent(s) in bioavailable form over extended periods of time. The duration of release from the sustained release formulations is dictated by the nature of the formulation and other factors discussed supra, such as for example proximity to bodily fluids and density of application of the formulations. However, sustained release formulations are designed to provide osteoinductive agents in the formulations at relatively consistent concentrations in bioavailable form over extended periods of time. Biodegradable sustained release polymers useful with the bioactive formulations are well known in the art and include, but are not limited to, polylactides, polyglycolides, polycaprolactones, polyanhydrides, polyamides, polyurethanes, polyesteramides, polyorthoesters, polydioxanones, polyacetals, polyketals, polycarbonates, polyorthocarbonates, polyphosphazenes, polyhydroxybutyrates, polyhydroxyvalerates, polyalkylene oxalates, polyalkylene succinates, poly(malic acid), poly(amino acids), polyvinylpyrrolidone, polyethylene glycol, polyhydroxycellulose, chitin, chitosan, poly(L-lactic acid), poly(lactide-co-glycolide), poly(hydroxybutyrate-co-valerate), and copolymers, terpolymers, or combinations or mixtures of the above materials. These materials preferably are compatible with the collagen and/or synthetic fibers used in the embodiments. The release profile of the biodegradable polymer can further be modified by inclusion of biostable polymers that influence the biodegradation rate of the polymer composition. Biostable polymers that could be incorporated into the biodegradable polymers, thereby influencing the rates of biodegradation, include but are not limited to silicones, polyesters, vinyl homopolymers and copolymers, acrylate homopolymers and copolymers, polyethers, and cellulosics.

The biodegradable polymers can be solid form polymers or alternatively can be liquid polymers that solidify in a reasonable time after application. Suitable liquid polymers formulations include, but are not limited to those polymer compositions disclosed in, for example, U.S. Pat. Nos. 5,744,153, 4,938,763, 5,278,201 and 5,278,202, the disclosures of each of which are herein incorporated by reference in their entireties. These patents disclose liquid polymer compositions that are useful as controlled drug-release compositions or as implants. The liquid prepolymer has at least one polymerizable ethylenically unsaturated group (e.g., an acrylic-ester-terminated prepolymer). If a curing agent is employed, the curing agent is typically added to the composition just prior to use. The prepolymer remains a liquid for a short period after the introduction of the curing agent. During this period the liquid delivery composition may be introduced into the orthopaedic implant device, e.g., via syringe. The mixture then solidifies to form a solid composition. The liquid polymer compositions may be administered to a patient in liquid form, and will then solidify or cure at the site of introduction to form a solid polymer composition. Biodegradable forms of the polymers are contemplated, and mixtures of biodegradable and biostable polymers are contemplated that affect the rate of biodegradation of the polymer.

Bioactive formulations further contemplate the use of aqueous and non-aqueous protic peptide formulations to maintain stability of the bioactive agents contained therein over extended periods of time. Non-limiting examples of aqueous and non-aqueous protic formulations useful for the long-term stability of bioactive agent(s) include those formulations provided in U.S. Pat. Nos. 5,916,582; 5,932,547, and 5,981,489, the disclosures of each of which are herein incorporated by reference in their entireties.

In another embodiment of the invention, the liquid compositions that are useful for the delivery of bioactive formulations in vivo include conjugates of the bioactive agent with a water-insoluble biocompatible polymer, with the dissolution of the resultant polymer-active agent conjugate in a biocompatible solvent to form a liquid polymer system. In addition, the liquid polymer system also may include a water-insoluble biocompatible polymer that is not conjugated to the bioactive agent. In one embodiment, these liquid compositions may be introduced into the body of a subject in liquid form. The liquid composition then solidifies or coagulates in situ to form a controlled release formulation where the bioactive agent is conjugated to the solid matrix polymer.

The bioactive formulations disclosed in the embodiments preferably include bioactive agents, and more preferably include osteoinductive and/or osteoconductive agents. Osteoinductive agents preferably are administered as components of the bioactive formulations as polypeptides or polynucleotides. Polynucleotide compositions of the osteoinductive agents include, but are not limited to, isolated Bone Morphogenetic Protein (BMP), Vascular Endothelial Growth Factor (VEGF), Connective Tissue Growth Factor (CTGF), Osteoprotegerin, Growth Differentiation Factors (GDFs), Cartilage Derived Morphogenic Proteins (CDMPs), Lim Mineralization Proteins (LMPs), and Transforming Growth Factor beta (TGF-β) polynucleotides. Polynucleotide compositions of the osteoinductive agents include, but are not limited to, gene therapy vectors harboring polynucleotides encoding the osteoinductive polypeptide of interest. Gene therapy methods require a polynucleotide which codes for the osteoinductive polypeptide operatively linked or associated to a promoter and any other genetic elements necessary for the expression of the osteoinductive polypeptide by the target tissue. Such gene therapy and delivery techniques are known in the art, (See, for example, International Publication No. WO90/11092, the disclosure of which is herein incorporated by reference in its entirety). Suitable gene therapy vectors include, but are not limited to, gene therapy vectors that do not integrate into the host genome. Alternatively, suitable gene therapy vectors include, but are not limited to, gene therapy vectors that integrate into the host genome.

In one embodiment, the polynucleotide is delivered in plasmid formulations. Plasmid DNA or RNA formulations refer to polynucleotide sequences encoding osteoinductive polypeptides that are free from any delivery vehicle that acts to assist, promote or facilitate entry into the cell, including viral sequences, viral particles, liposome formulations, lipofectin or precipitating agents and the like. Optionally, gene therapy compositions can be delivered in liposome formulations and lipofectin formulations, which can be prepared by methods well known to those skilled in the art. General methods are described, for example, in U.S. Pat. Nos. 5,593,972, 5,589,466, and 5,580,859, the disclosures of which are herein incorporated by reference in their entireties.

Gene therapy vectors further comprise suitable adenoviral vectors including, but not limited to for example, those described in Kozarsky and Wilson, Curr. Opin. Genet. Devel.,3:499-503 (1993); Rosenfeld et al., Cell, 68:143-155 (1992); Engelhardt et al., Human Genet. Ther., 4:759-769 (1993); Yang et al., Nature Genet., 7:362-369 (1994); Wilson et al., Nature, 365:691-692 (1993); and U.S. Pat. No. 5,652,224, which are herein incorporated by reference in their entireties.

Polypeptide compositions of the isolated osteoinductive agents include, but are not limited to, isolated Bone Morphogenetic Protein (BMP), Vascular Endothelial Growth Factor (VEGF), Connective Tissue Growth Factor (CTGF), Osteoprotegerin, Growth Differentiation Factors (GDFs), Cartilage Derived Morphogenic Proteins (CDMPs), Lim Mineralization Proteins (LMPs), and Transforming Growth Factor beta (TGF-β707 ) polypeptides. Polypeptide compositions of the osteoinductive agents include, but are not limited to, full length proteins, fragments and variants thereof. In a preferred embodiment, polypeptide fragments of the osteoinductive agents are propeptide forms of the isolated full length polypeptides. In a particularly preferred embodiment, polypeptide fragments of the osteoinductive agents are mature forms of the isolated full length polypeptides. Also preferred are the polynucleotides encoding the propeptide and mature polypeptides of the osteoinductive agents.

Variants of the isolated osteoinductive agents include, but are not limited to, polypeptide variants that are designed to increase the duration of activity of the osteoinductive agent in vivo. Preferred embodiments of variant osteoinductive agents include, but are not limited to, full length proteins or fragments thereof that are conjugated to polyethylene glycol (PEG) moieties to increase their half-life in vivo (also known as pegylation). Methods of pegylating polypeptides are well known in the art (See, e.g., U.S. Pat. No. 6,552,170 and European Pat. No. 0,401,384 as examples of methods of generating pegylated polypeptides).

In another embodiment, the isolated osteoinductive agent(s) are provided in the bioactive formulation(s) as fusion proteins. In one embodiment, the osteoinductive agent(s) are available as fusion proteins with the Fc portion of human IgG. In another embodiment, the osteoinductive agent(s) are available as hetero- or homodimers or multimers. Examples of preferred fusion proteins include, but are not limited to, ligand fusions between mature osteoinductive polypeptides and the F_{c} portion of human Immunoglobulin G (IgG). Methods of making fusion proteins and constructs encoding the same are well known in the art.

Isolated osteoinductive agents that are included within the bioactive formulations preferably are sterile. In a non-limiting method, sterility is readily accomplished for example by filtration through sterile filtration membranes (e.g., 0.2 micron membranes or filters).

In one embodiment, the composite implant is packaged without impregnated bioactive formulations, such as for example where the composite implant comprises a porous substrate into which the bioactive formulations are subsequently impregnated. In such a situation, osteoinductive agents generally are placed into a container having a sterile access port, for example, a solution bag or vial having a stopper pierceable by a hypodermic injection needle. In one embodiment, osteoinductive agents and prepared bioactive formulations are stored in separate containers, for example, sealed ampoules or vials, as an aqueous solution or as a lyophilized formulation for reconstitution. As an example of a lyophilized formulation, 10-ml vials are filled with 5 ml of sterile-filtered 1% (w/v) aqueous osteoinductive agent solution, and the resulting mixture is lyophilized. The osteoinductive agent is prepared by reconstituting the lyophilized agent prior to administration in an appropriate solution, admixed with the prepared bioactive formulations and administered to the composite implant prior to or concurrent with implantation into a patient.

As one of skill in the art will recognize, the concentrations of osteoinductive agent can be variable based on the desired length or degree of osteoinduction. Similarly, one of skill in the art will understand that the duration of sustained release can be modified by the manipulation of the compositions comprising the sustained release formulation, such as for example, modifying the percent of biostable polymers found within a sustained release formulation, microencapsulation of the formulation within polymers, including polymers having varying degradation times and characteristics, and layering the formulation in varying thicknesses in one or more degradable polymers. These sustained release formulations can therefore be designed to provide customized time release of factors that simulate the natural healing process.

Another method to provide liquid compositions that are useful for the delivery of osteoinductive agents in vivo and permit the initial burst of bioactive agent to be controlled more effectively than previously possible is to conjugate the active agent with a water-insoluble biocompatible polymer and dissolve the resultant polymer-active agent conjugate in a biocompatible solvent to form a liquid polymer system similar to that described in U.S. Pat. Nos. 4,938,763, 5,278,201 and 5,278,202, the disclosures of each of which are incorporated by reference herein in their entireties. The water-insoluble biocompatible polymers may be those described in the above patents or related copolymers. In addition, the liquid polymer system also may include a water-insoluble biocompatible polymer that is not conjugated to the active agent. In one embodiment, these liquid compositions may be introduced into the body of a subject in liquid form. The liquid composition then solidifies or coagulates in situ to form a controlled release implant where the active agent is conjugated to the solid matrix polymer.

The bioactive formulation employed to form the controlled release implant in situ may be a liquid delivery composition that includes a biocompatible polymer that is substantially insoluble in aqueous medium, an organic solvent which is miscible or dispersible in aqueous medium, and the controlled release component. The biocompatible polymer is substantially dissolved in the organic solvent. The controlled release component may be either dissolved, dispersed or entrained in the polymer/solvent solution. In a preferred embodiment, the biocompatible polymer is biodegradable and/or bioerodable.

Bioactive formulations optionally further comprise de-mineralized bone matrix compositions (hereinafter “DBM” compositions), bone marrow aspirate, bone marrow concentrate, or combinations or permutations of any of the same. Methods for producing DBM are well known in the art, and DBM may be obtained following the teachings of O'Leary et al. (U.S. Pat. No. 5,073,373) or by obtaining commercially available DBM formulations such as, for example, AlloGro® available from suppliers such as AlloSource® (Centennial, Colo.). Methods of obtaining bone marrow aspirates as well as devices facilitating extraction of bone marrow aspirate are well known in the art and are described, for example, by Turkel et al. in U.S. Pat. No. 5,257,632.

Bioactive formulations optionally further comprise antibiotics that are administered with the isolated osteoinductive agent. As discussed by Vehmeyer et al., the possibility exists that bacterial contamination can occur for example due to the introduction of contaminated allograft tissue from living donors. Vehmeyer, S B, et al., Acta Orthop Scand., 73(2): 165-169 (2002). Antibiotics also may be co-administered with the bioactive formulations to prevent infection by obligate or opportunistic pathogens that are introduced to the patient during implant surgery.

Antibiotics useful with the bioactive formulations include, but are not limited to, amoxicillin, beta-lactamases, aminoglycosides, beta-lactam (glycopeptide), clindamycin, chloramphenicol, cephalosporins, ciprofloxacin, erythromycin, fluoroquinolones, macrolides, metronidazole, penicillins, quinolones, rapamycin, rifampin, streptomycin, sulfonamide, tetracyclines, trimethoprim, trimethoprim-sulfamethoxazole, and vancomycin. In addition, one skilled in the art of implant surgery or administrators of locations in which implant surgery occurs may prefer the introduction of one of more the above-recited antibiotics to account for nosocomial infections or other factors specific to the location where the implant surgery is conducted. Accordingly, the bioactive formulations contemplate that one or more of the antibiotics recited supra, and any combination of one or more of the same antibiotics, may be included therein.

The bioactive formulations optionally further comprise immunosuppressive agents, particularly in circumstances where allograft compositions are administered to the patient. Suitable immunosuppressive agents that may be administered in combination with the bioactive formulations include, but are not limited to, steroids, cyclosporine, cyclosporine analogs, cyclophosphamide, methylprednisone, prednisone, azathioprine, FK-506, 15-deoxyspergualin, and other immunosuppressive agents that act by suppressing the function of responding T cells. Other immunosuppressive agents that may be administered in combination with the osteoinductive formulations of the invention include, but are not limited to, prednisolone, methotrexate, thalidomide, methoxsalen, rapamycin, leflunomide, mizoribine (bredinin™), brequinar, deoxyspergualin, and azaspirane (SKF 105685), Orthoclone OKT™ 3 (muromonab-CD3). Sandimmune™, Neoral™, Sangdya™ (cyclosporine), Prograf™ (FK506, tacrolimus), Cellcept™ (mycophenolate motefil, of which the active metabolite is mycophenolic acid), Imuran™ (azathioprine), glucocorticosteroids, adrenocortical steroids such as Deltasone™ (prednisone) and Hydeltrasol™ (prednisolone), Folex™ and Mexate™ (methotrexate), Oxsoralen-Ultra™ (methoxsalen) and Rapamuen™ (sirolimus).

The bioactive formulations may optionally further comprise a carrier vehicle such as water, saline, Ringer's solution, calcium phosphate based carriers, or dextrose solution. Non-aqueous vehicles such as fixed oils and ethyl oleate are also useful herein, as well as liposomes.

The bioactive formulations further optionally include substances that enhance isotonicity and chemical stability. Such materials are non-toxic to patients at the dosages and concentrations employed, and include buffers such as phosphate, citrate, succinate, acetic acid, and other organic acids or their salts; antioxidants such as ascorbic acid; low molecular weight (less than about ten residues) polypeptides, e.g., polyarginine or tripeptides; proteins, such as serumalbumin, gelatin, or immunoglobulins; amino acids, such as glycine, glutamic acid, aspartic acid, or arginine; monosaccharides, disaccharides, and other carbohydrates including cellulose or its derivatives, glucose, mannose, or dextrins; chelating agents such as EDTA; sugaralcohols such as mannitol or sorbitol; counterions such as sodium; and/or nonionicsurfactants such as polysorbates, poloxamers, or PEG.

Bioactive formulations further comprise isolated osteoinductive agents. Isolated osteoinductive agents promote the in-growth of endogenous bone into, around, or on the spinal implant device, or alternatively promote the growth of connective tissue, vascular tissue, or aid in preventing resorption of bone tissue by osteoclasts. Isolated osteoinductive agents are available as polypeptides or polynucleotides. Isolated osteoinductive agents preferably comprise full length proteins and fragments thereof, as well as polypeptide variants or mutants of the isolated osteoinductive agents provided herein.

Recombinantly expressed proteins may be in native forms, truncated analogs, muteins, fusion proteins, and other constructed forms capable of inducing bone, cartilage, or other types of tissue formation as demonstrated by in vitro and ex vivo bioassays and in vivo implantation in mammals, including humans.

The polynucleotides and polypeptides useful in the bioactive formulations preferably have at least 95% homology, more preferably 97%, and even more preferably 99% homology to the isolated osteoinductive agent polynucleotides and polypeptides provided herein. Typical bioactive formulations comprise isolated osteoinductive agent at concentrations of from about 0.1 mg/ml to 100 mg/ml, preferably 1-10 mg/ml, at a pH of about 3 to 8.

In one embodiment, the isolated osteoinductive agents include one or more members of the family of Bone Morphogenetic Proteins (“BMPs”). BMPs are a class of proteins thought to have osteoinductive or growth-promoting activities on endogenous bone tissue, or function as pro-collagen precursors. Known members of the BMP family include, but are not limited to, BMP-1, BMP-2, BMP-3, BMP-4, BMP-5, BMP-6, BMP-7, BMP-8, BMP-9, BMP-10, BMP-11, BMP-12, BMP-13, BMP-15, BMP-16, BMP-17, and BMP-18.

BMPs useful as isolated osteoinductive agents include, but are not limited to, the following BMPs:

BMP-1 polynucleotides and polypeptides, as well as mature BMP-1 polypeptides and polynucleotides encoding the same;

BMP-2 polynucleotides and polypeptides, as well as mature BMP-2 polypeptides and polynucleotides encoding the same;

BMP-3 polynucleotides and polypeptides, as well as mature BMP-3 polypeptides and polynucleotides encoding the same;

BMP-4 polynucleotides and polypeptides, as well as mature BMP-4 polypeptides and polynucleotides encoding the same;

BMP-5 polynucleotides and polypeptides, as well as mature BMP-5 polypeptides and polynucleotides encoding the same;

BMP-6 polynucleotides and polypeptides, as well as mature BMP-6 polypeptides and polynucleotides encoding the same;

BMP-7 polynucleotides and polypeptides, as well as mature BMP-7 polypeptides and polynucleotides encoding the same;

BMP-8 polynucleotides and polypeptides, as well as mature BMP-8 polypeptides and polynucleotides encoding the same;

BMP-9 polynucleotides and polypeptides, as well as mature BMP-9 polypeptides and polynucleotides encoding the same;

BMP-10 polynucleotides and polypeptides, as well as mature BMP-10 polypeptides and polynucleotides encoding the same;

BMP-11 polynucleotides and polypeptides, as well as mature BMP-11 polypeptides and polynucleotides encoding the same;

BMP-12 polynucleotides and polypeptides, as well as mature BMP-12 polypeptides and polynucleotides encoding the same;

BMP-13 polynucleotides and polypeptides, as well as mature BMP-13 polypeptides and polynucleotides encoding the same;

BMP-15 polynucleotides and polypeptides, as well as mature BMP-15 polypeptides and polynucleotides encoding the same;

BMP-16 polynucleotides and polypeptides, as well as mature BMP-16 polypeptides and polynucleotides encoding the same;

BMP-17 polynucleotides and polypeptides, as well as mature BMP-17 polypeptides and polynucleotides encoding the same; and

BMP-18 polynucleotides and polypeptides, as well as mature BMP-18 polypeptides and polynucleotides encoding the same.

BMPs utilized as osteoinductive agents comprise, or alternatively consist of, one or more of BMP-1; BMP-2; BMP-3; BMP-4; BMP-5; BMP-6; BMP-7; BMP-8; BMP-9; BMP-10; BMP-11; BMP-12; BMP-13; BMP-15; BMP-16; BMP-17; and BMP-18; as well as any combination of one or more of these BMPs, including full length BMPs or fragments thereof, or combinations thereof, either as polypeptides or polynucleotides encoding the polypeptide fragments of all of the recited BMPs. The isolated BMP osteoinductive agents may be administered as polynucleotides, polypeptides, or combinations of both. In a particularly preferred embodiment of the invention, isolated osteoinductive agents comprise, or alternatively consist of, BMP-2 polynucleotides or polypeptides or mature fragments of the same.

In another embodiment, isolated osteoinductive agents include osteoclastogenesis inhibitors to inhibit bone resorption of the bone tissue surrounding the site of implantation of the spinal implant device by osteoclasts. Osteoclast and Osteoclastogenesis inhibitors include, but are not limited to, Osteoprotegerin polynucleotides and polypeptides, as well as mature Osteoprotegerin polypeptides and polynucleotides encoding the same. Osteoprotegerin is a member of the TNF-receptor superfamily and is an osteoblast-secreted decoy receptor that functions as a negative regulator of bone resorption. This protein specifically binds to its ligand, osteoprotegerin ligand (TNFSF11/OPGL), both of which are key extracellular regulators of osteoclast development.

Osteoclastogenesis inhibitors further include, but are not limited to, chemical compounds such as bisphosphonate, 5-lipoxygenase inhibitors such as those described in U.S. Pat. Nos. 5,534,524 and 6,455,541 (the contents of which are herein incorporated by reference in their entierties), heterocyclic compounds such as those described in U.S. Pat. No. 5,658,935 (herein incorporated by reference in its entirety), 2,4-dioxoimidazolidine and imidazolidine derivative compounds such as those described in U.S. Pat. Nos. 5,397,796 and 5,554,594 (the contents of which are herein incorporated by reference in their entireties), sulfonamide derivatives such as those described in U.S. Pat. No. 6,313,119 (herein incorporated by reference in its entierty), and acylguanidine compounds such as those described in U.S. Pat. No. 6,492,356 (herein incorporated by reference in its entirety).

In another embodiment, isolated osteoinductive agents include one or more members of the family of Connective Tissue Growth Factors (“CTGFs”). CTGFs are a class of proteins thought to have growth-promoting activities on connective tissues. Known members of the CTGF family include, but are not limited to, CTGF-1, CTGF-2, and CTGF-4.

CTGFs useful as isolated osteoinductive agents include, but are not limited to, the following CTGFs:

CTGF-1 polynucleotides and polypeptides, as well as mature CTGF-1 polypeptides and polynucleotides encoding the same.

CTGF-2 polynucleotides and polypeptides, as well as mature CTGF-2 polypeptides and polynucleotides encoding the same.

CTGF-4 polynucleotides and polypeptides, as well as mature CTGF-4 polypeptides and polynucleotides encoding the same.

In another embodiment, isolated osteoinductive agents include one or more members of the family of Vascular Endothelial Growth Factors (“VEGFs”). VEGFs are a class of proteins thought to have growth-promoting activities on vascular tissues. Known members of the VEGF family include, but are not limited to, VEGF-A, VEGF-B, VEGF-C, VEGF-D and VEGF-E.

VEGFs useful as isolated osteoinductive agents include, but are not limited to, the following VEGFs:

VEGF-A polynucleotides and polypeptides, as well as mature VEGF-A polypeptides and polynucleotides encoding the same.

VEGF-B polynucleotides and polypeptides, as well as mature VEGF-B polypeptides and polynucleotides encoding the same.

VEGF-C polynucleotides and polypeptides, as well as mature VEGF-C polypeptides and polynucleotides encoding the same.

VEGF-D polynucleotides and polypeptides, as well as mature VEGF-D polypeptides and polynucleotides encoding the same.

VEGF-E polynucleotides and polypeptides, as well as mature VEGF-E polypeptides and polynucleotides encoding the same.

In another embodiment, isolated osteoinductive agents include one or more members of the family of Transforming Growth Factor-beta genes (“TGF-βs”). TGF-βs are a class of proteins thought to have growth-promoting activities on a range of tissues, including connective tissues. Known members of the TGF-β family include, but are not limited to, TGF-β-1, TGF-β-2, and TGF-β-3.

TGF-βs useful as isolated osteoinductive agents include, but are not limited to, the following TGF-βs:

TGF-β-1 polynucleotides and polypeptides, as well as mature TGF-β-1 polypeptides and polynucleotides encoding the same.

TGF-β-2 polynucleotides and polypeptides, as well as mature TGF-β-2 polypeptides and polynucleotides encoding the same.

TGF-β-3 polynucleotides and polypeptides, as well as mature TGF-β-3 polypeptides and polynucleotides encoding the same.

In another embodiment, isolated osteoinductive agents include one or more Growth Differentiation Factors (“GDFs”). Known GDFs include, but are not limited to, GDF-1, GDF-2, GDF-3, GDF-7, GDF-10, GDF-11, and GDF-15.

GDFs useful as isolated osteoinductive agents include, but are not limited to, the following GDFs:

GDF-1 polynucleotides and polypeptides corresponding to GenBank Accession Numbers M62302, AAA58501, and AAB94786, as well as mature GDF-1 polypeptides and polynucleotides encoding the same.

GDF-2 polynucleotides and polypeptides corresponding to GenBank Accession Numbers BC069643, BC074921, Q9UK05, AAH69643, and AAH74921, as well as mature GDF-2 polypeptides and polynucleotides encoding the same.

GDF-3 polynucleotides and polypeptides corresponding to GenBank Accession Numbers AF263538, BC030959, AAF91389, AAQ89234, and Q9NR23, as well as mature GDF-3 polypeptides and polynucleotides encoding the same.

GDF-7 polynucleotides and polypeptides corresponding to GenBank Accession Numbers AB158468, AF522369, AAP97720, and Q7Z4P5, as well as mature GDF-7 polypeptides and polynucleotides encoding the same.

GDF-10 polynucleotides and polypeptides corresponding to GenBank Accession Numbers BC028237 and AAH28237, as well as mature GDF-10 polypeptides and polynucleotides encoding the same.

GDF-11 polynucleotides and polypeptides corresponding to GenBank Accession Numbers AF100907, NP_{—}005802 and 095390, as well as mature GDF-11 polypeptides and polynucleotides encoding the same.

GDF-15 polynucleotides and polypeptides corresponding to GenBank Accession Numbers BC008962, BC000529, AAH00529, and NP_{—}004855, as well as mature GDF-15 polypeptides and polynucleotides encoding the same.

In another embodiment, isolated osteoinductive agents include Cartilage Derived Morphogenic Protein (CDMP) and Lim Mineralization Protein (LMP) polynucleotides and polypeptides. Known CDMPs and LMPs include, but are not limited to, CDMP-1, CDMP-2, LMP-1, LMP-2, and LMP-3.

CDMPs and LMPs useful as isolated osteoinductive agents include, but are not limited to, the following CDMPs and LMPs:

CDMP-1 polynucleotides and polypeptides corresponding to GenBank Accession Numbers NM_{—}000557, U13660, NP_{—}000548 and P43026, as well as mature CDMP-1 polypeptides and polynucleotides encoding the same.

CDMP-2 polypeptides corresponding to GenBank Accession Numbers and P55106, as well as mature CDMP-2 polypeptides.

LMP-1 polynucleotides and polypeptides corresponding to GenBank Accession Numbers AF345904 and AAK30567, as well as mature LMP-1 polypeptides and polynucleotides encoding the same.

LMP-2 polynucleotides and polypeptides corresponding to GenBank Accession Numbers AF345905 and AAK30568, as well as mature LMP-2 polypeptides and polynucleotides encoding the same.

LMP-3 polynucleotides and polypeptides corresponding to GenBank Accession Numbers AF345906 and AAK30569, as well as mature LMP-3 polypeptides and polynucleotides encoding the same.

In another embodiment, isolated osteoinductive agents include one or more members of any one of the families of Bone Morphogenetic Proteins (BMPs), Connective Tissue Growth Factors (CTGFs), Vascular Endothelial Growth Factors (VEGFs), Osteoprotegerin or any of the other osteoclastogenesis inhibitors, Growth Differentiation Factors (GDFs), Cartilage Derived Morphogenic Proteins (CDMPs), Lim Mineralization Proteins (LMPs), and Transforming Growth Factor-betas (TGF-βs), as well as mixtures and combinations thereof.

In another embodiment, the one or more isolated osteoinductive agents useful in the bioactive formulation are selected from the group consisting of BMP-1, BMP-2, BMP-3, BMP-4, BMP-5, BMP-6, BMP-7, BMP-8, BMP-9, BMP-10, BMP-11, BMP-12, BMP-13, BMP-15, BMP-16, BMP-17, BMP-18, and any combination thereof; CTGF-1, CTGF-2, CGTF-3, CTGF-4, and any combination thereof; VEGF-A, VEGF-B, VEGF-C, VEGF-D, VEGF-E, and any combination thereof; GDF-1, GDF-2, GDF-3, GDF-7, GDF-10, GDF-11, GDF-15, and any combination thereof; CDMP-1, CDMP-2, LMP-1, LMP-2, LMP-3, and any combination thereof; Osteoprotegerin; TGF-b-1, TGF-b-2, TGF-b-3, and any combination thereof; and any combination of one or more members of these groups.

Embodiments of the invention further include methods of making the composite spinal implants described herein. Methods for producing the composite spinal implants are well known in the art and are largely dictated by the particular spinal implant device that will be implanted. The composite implants described herein, however, also include collagen and/or synthetic fibers that are impregnated with the bioactive formulations described above. As stated previously, the bioactive formulations may be impregnated prior to, during, or after formation of the implant. Perferably, the bioactive formulations are impregnated into the spinal implant after it has been formed.

The spinal implants can be formed using any techniques commonly employed in forming implants. Preferably, the collagen and/or synthetic fibers are formed into the desired shape using a mold or other mold-like apparatus. Heat and/or pressure preferably are used to assist in formation of the shaped article. Methods of suturing or annealing collagen to itself are described in, for example, U.S. Pat. No. 6,719,795, the disclosure of which is incorporated by reference herein in its entirety. Methods of forming implants from synthetic fibers also are known and described in, for example, U.S. Pat. No. 5,348,026, the disclosure of which is incorporated by reference herein in its entirety. Other methods of fabricating implants with a synthetic fiber or collagen are disclosed above.

The composite spinal implants can be manufactured by supplying a collagen and/or synthetic fiber material. These materials may optionally be admixed together with one or a combination of biocompatible metals, metal alloys, or ceramics to provide a moldable implant material. The moldable implant material then is formed into the desired implant shape and optionally further treated to create the composite implant. Optional further treatment includes sintering, heating, cooling, immersion in fluids or gases, as well as surface treatments to roughen or make porous the surface, as described above.

To form the composite implant in its desired shape, any number of methods can be used. Tape casting of ceramics and collagen and/or synthetic fibers can be used to form a ceramic composite, the tape material manually formed or pressed into a mold, and then the material sintered. Pore formers may be present to provide a porous ceramic composite, which then may be impregnated with the bioactive formulation. The ceramic material and collagen and/or synthetic fibers can be provided as powders or granules, and pressed using hot isostatic pressing or other compression forming techniques to form an implant having the desired shape. Die casting, injection molding, or extrusion molding can be used if metals, metal alloys, or biocompatible polymers are used to form the composite material together with the collagen and/or synthetic fiber material. Skilled artisans are aware of the myriad implant formation techniques, and are capable of using any of these techniques to form the composite implants of the embodiments, using the guidelines provided herein.

It is especially preferred in the embodiments to impregnate the collagen and/or synthetic fiber contained in the composite implant with the bioactive formulation after formation of the implant. Skilled artisans will appreciate, however, that the collagen and/or synthetic fiber may be impregnated prior to or during implant formation. The bioactive formulation may be applied to the composite implant device using any of a number of methods, such as for example by spraying or brushing the bioactive formulation onto the composite implant device. The bioactive formulation also may be applied to the composite spinal implant device by immersing the device in a solution comprising the bioactive formulation.

In addition to, or as a substitute of the bioactive formulations described herein, embodiments may utilize vectors containing the polynucleotide of the osteoconductive or osteoinductive agent, host cells, and the production of polypeptides by recombinant techniques. These embodiments provide the osteoconductive or osteoinductive agent in a bioavailable form in vivo. The vector may be, for example, a phage, plasmid, viral, or retroviral vector. Retroviral vectors may be replication competent or replication defective. In the latter case, viral propagation generally will occur only in complementing host cells.

The polynucleotides may be joined to a vector containing a selectable marker for propagation in a host. Generally, a plasmid vector is introduced in a precipitate, such as a calcium phosphate precipitate, or in a complex with a charged lipid. If the vector were a virus, it may be packaged in vitro using an appropriate packaging cell line and then transduced into host cells. Useful vectors include, but are not limited to, plasmids, bacteriophage, insect and animal cell vectors, retroviruses, cosmids, and other single and double-stranded viruses.

The polynucleotide insert should be operatively linked to an appropriate promoter, such as the phage lambda PL promoter, the E. coli lac, trp, phoA and tac promoters, the SV40 early and late promoters and promoters of retroviral LTRs, to name a few. Other suitable promoters will be known to the skilled artisan. The expression constructs will further contain sites for transcription initiation, termination; origin of replication sequence, and, in the transcribed region, a ribosome binding site for translation. The coding portion of the transcripts expressed by the constructs will preferably include a translation initiating codon at the beginning and a termination codon (UAA, UGA or UAG) appropriately positioned at the end of the polypeptide to be translated. The expression construct may further contain sequences such as enhancer sequences, efficient RNA processing signals such as splicing and polyadenylation signals, sequences that enhance translation efficiency, and sequences that enhance protein secretion.

Expression systems and methods of producing osteoinductive agents, such as recombinant proteins or protein fragments, are well known in the art. For example, methods of producing recombinant proteins or fragments thereof using bacterial, insect or mammalian expression systems are well known in the art. (See, e.g., Molecular Biotechnology: Principles and Applications of Recombinant DNA, B. R. Glick and J. Pasternak, and M. M. Bendig, Genetic Engineering, 7, pp. 91-127 (1988), for a general discussion of recombinant protein production).

The expression vectors will preferably include at least one selectable marker. Such markers include dihydrofolate reductase, G418 or neomycin resistance for eukaryotic cell culture and tetracycline, kanamycin or ampicillin resistance genes for culturing in E. coli and other bacteria. Representative examples of appropriate hosts include, but are not limited to, bacterial cells, such as E. coli, Streptomyces and Salmonella typhimurium cells; fungal cells, such as Pichia and other yeast cells; insect cells such as Drosophila S2 and Spodoptera Sf9 and Sf21 cells; animal cells such as CHO, COS, 293, and Bowes melanoma cells; and plant cells. Appropriate culture mediums and conditions for the above-described host cells are known in the art.

Examples of vectors for use in prokaryotes include pQE30Xa and other pQE vectors available in pQE expression systems available from QIAGEN, Inc. (Valencia, Calif.); pBluescript vectors, Phagescript vectors, pNH8A, pNH16a, pNH18A, pNH46A, available from Stratagene Cloning Systems, Inc. (La Jolla, Calif.); and Champion™, T7, and pBAD vectors available from Invitrogen (Carlsbad, Calif.). Other suitable vectors will be readily apparent to the skilled artisan.

Introduction of the construct into the host cell can be effected by calcium phosphate transfection, DEAE-dextran mediated transfection, cationic lipid-mediated transfection, electroporation, transduction, infection, or other methods. Such methods are described in many standard laboratory manuals, such as Davis et al., Basic Methods In Molecular Biology (198). The host cells, and expression vectors preferably are impregnated into the collagen and/or synthetic fibers using any of the above-described techniques.

A polypeptide useful in the bioactive formulation can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography (“HPLC”) is employed for purification.

In another embodiment, osteoinductive agents can be produced using bacterial lysates in cell-free expression systems that are well known in the art. Commercially available examples of cell-free protein synthesis systems include the EasyXpress System from Qiagen, Inc. (Valencia, Calif.).

Polypeptides of the present invention also can be recovered from the following: products of chemical synthetic procedures; and products produced by recombinant techniques from a prokaryotic or eukaryotic host, including, for example, bacterial, yeast, higher plant, insect, and mammalian cells.

Depending upon the host employed in a recombinant production procedure, the polypeptides may be glycosylated or may be non-glycosylated. In addition, polypeptides also may include an initial modified methionine residue, in some cases as a result of host-mediated processes. Thus, it is well known in the art that the N-terminal methionine encoded by the translation initiation codon generally is removed with high efficiency from any protein after translation in all eukaryotic cells. While the N-terminal methionine on most proteins also is efficiently removed in most prokaryotes, for some proteins, this prokaryotic removal process is inefficient, depending on the nature of the amino acid to which the N-terminal methionine is covalently linked.

The osteoinductive agents also may be isolated from natural sources of polypeptide. Osteoinductive agents may be purified from tissue sources, preferably mammalian tissue sources, using conventional physical, immunological and chemical separation techniques known to those of skill in the art. Appropriate tissue sources for the desired osteoinductive agents are known or are available to those of skill in the art.

The bioactive formulation of the embodiments also may include cells, such as intervertebral disc cells that may have been removed from the nucleus pulposus of the patient prior to insertion of the implant device. The cells also may include other useful cells including bone cells, stem cells, nerve stem cells, chondrocytic cells, blood cells, plama cells (optionally combined with thrombin and/or calcium chloride), and the like. Use of cells cultured from the patient or elsewhere in assisting in spine surgery is described in, for example, U.S. Pat. Nos. 6,685,695, 6,454,804, 6,419,702, 6,340,369, 6,569,204, and U.S. Patent Publication No. 2002/0032155, the disclosures of which are incorporated by reference herein in their entirety. Other documents disclosing the use of cultured cells, which optionally are genetically modified prior to use, include Wehling, Peter, et al., “Transfer of Genes to Chondrocytic Cells of the Lumbar Spine: Proposal for a Treatment Strategy of Spinal Disorders by Local Gene Therapy,” Spine, Vol. 22, pp 1092-1097 (May 15, 1997); Nishida, Kotaro, et al., “Adenovirus-Mediated Gene Transfer to Nucleus Pulposus Cells: Implications for the Treatment of Intervertebral Disc Degeneration,” Spine, Vol. 23, pp 2437-2442 (Nov. 15, 1998); and Nishida, Kotaro, et al., “Modulation of the Biologic Activity of the Rabbit Intervertebral Disc by Gene Therapy: An In Vivo Study of Adenovirus-Mediated Transfer of the Human Transforming Growth Factor β1 Encoding Gene,” Spine, Vol. 24, pp 2419-25 (Nov. 23, 1999). Any of the techniques described in these documents can be used to harvest cells, preferably intravertebral nucleus cells, optionally genetically modifying the cells, and then impregnated the cells into or on the surface of a composite implant containing collagen and/or synthetic fibers.

Copmposite spinal implant devices of the embodiments are useful in enhancing the rate of ingrowth of endogenous bone into the site of implantation of the spinal implant device. The increased rate of endogenous bone ingrowth results in an increased rate and degree of implant adhesion to the remaining endogenous bone, connective tissue and related tissues. The increased rate of endogenous bone ingrowth decreases the amount of time necessary for the implant to achieve stability in the patient, thereby decreasing the recovery time of the implant patient. The endogenous bone ingrowth additionally enhances the stability of the implant by helping to minimize the ability of bodily fluids or any wear debris from impacting the interface of the implant and endogenous bone, which could play a role in failure of the implant. The implant device also mitigates the effects of any osteolysis that may occur in endogenous bone tissue surrounding the implant, particularly endogenous bone tissue that is in close proximity to joints or other locations of extensive motion and wear in the patient (e.g., discs and facet joints).

Another embodiment includes a method of performing a spinal surgery on a patient whereby the area of the spine is accessed and cleaned, preferably using minimally invasive techniques. The composite spinal implant then is inserted and positioned in the appropriate area of the spine, and the incision used for access and implantation, which may the same or different incision(s), is surgically closed. If the composite implant is a composite disc replacement, or spinal fusion device, the method would preferably include providing access to the nucleus through the annulus, resecting at least a portion of the nucleus pulposus, inserting the implant, closing the access through the annulus, and closing the skin incision(s). Other spinal surgeries that do not involve nucleus replacement or resection also are included, such as correction of spinal deformities, like scoliosis and spondylolisthesis. Rods, screws, and plates can be made of the composite implants described herein, and implanted using known surgical techniques.

In an additional embodiment, the composite spinal implants are packaged in kits under sterile conditions, and may be prepared as either “wet” kits or “dry” kits. In both types of kits, these kits comprise a composite implant including a collagen and/or synthetic fiber. The term “wet” as it modifies “kits” denotes kits comprising a composite spinal implant that alread is impregnated with the bioactive formulations prior to packaging, such that the composite spinal implant device impregnated with the bioactive formulations are prepared for implantation upon opening of the kit. Wet kits optionally further comprise antibiotics and metal ion chelating agents such as EDTA.

The term “dry” as it modifies “kits” denotes kits comprising a composite spinal implant device that is not impregnated with the bioactive formulations prior to packaging. In one embodiment, “dry” kits comprise the composite spinal implant device as one component of the kit. The dry kit further comprises the bioactive formulations packaged under separate container(s) in the dry kit. The bioactive formulations may be applied to the composite spinal implant device prior to implantation in the patient, for example by immersion of the composite spinal implant device in a solution of the bioactive composition. Alternatively, the bioactive composition may be applied with a sterile brush or other appropriate application device. The bioactive formulations also may be applied by dripping the bioactive formulations onto the composite spinal implant device through the use of a sterile eye dropper or similar applicator.

The kits described herein may further contemplate the addition of a sterile applicator, such as for example, a brush or dropper devices (e.g., eye droppers). The kits further optionally comprise instructions for the preparation and administration of the osteoinductive formulations and the orthopaedic device. The kits further may include one or more surgical instruments useful in inserting the composite spinal implant device, or in performing the requisite spinal surgery to implant the composite spinal implant.

The embodiments described herein have been described with reference to particularly preferred embodiment, but may be practiced in ways other than those particularly described in the foregoing description. Numerous modifications and variations of the embodiments are possible in light of the above teachings and, therefore, are within the scope of the appended claims.

What is claimed is:

1. A composite spinal implant device useful for promoting in-growth of bone and vascular tissue, comprising a composite spinal implant device comprising at least one of collagen or a synthetic fiber, at least a portion of which is impregnated with a bioactive formulation.

2. The implant device as claimed in claim 1, wherein the bioactive formulation comprises an osteoclastogenesis inhibitor.

3. The implant device of claim 1, wherein the bioactive formulation further comprises one or more isolated osteoinductive agents.

4. The implant device of claim 3, wherein the one or more isolated osteoinductive agents is selected from the group consisting of one or more BMPs, one or more VEGFs, one or more CTGFs, one or more GDFs, one or more CDMPs, one or more LMPs, one or more TGF-β, and any combination thereof.

5. The implant device of claim 3, wherein the one or more isolated osteoinductive agents are selected from the group consisting of:
a) BMP-1, BMP-2, BMP-3, BMP-4, BMP-5, BMP-6, BMP-7, BMP-8, BMP-9, BMP-10, BMP-11, BMP-12, BMP-13, BMP-15, BMP-16, BMP-17, BMP-18, and any combination thereof;
b) CTGF-1, CTGF-2, CGTF-3, CTGF-4, and any combination thereof;
c) VEGF-A, VEGF-B, VEGF-C, VEGF-D, VEGF-E, and any combination thereof;
d) GDF-1, GDF-2, GDF-3, GDF-7, GDF-10, GDF-11, GDF-15, and any combination thereof;
e) CDMP-1, CDMP-2, LMP-1, LMP-2, LMP-3, and any combination thereof;
f) TGF-β-1, TGF-β-2, TGF-β-3, and any combination thereof; and
g) any combination of one or more members of these groups.

6. The implant device of claim 3, wherein the bioactive formulation comprises a sustained-release formulation.

7. The implant device of claim 3, wherein the bioactive formulation further comprises one or more additives selected from the group consisting of antibiotics, demineralized bone matrix, bone marrow aspirate, bone marrow concentrate, immunosuppressives, and combinations or mixtures thereof.

8. The implant device of claim 3, wherein the osteoinductive formulation further comprises a carrier.

9. The implant device of claim 2, wherein theosteoclastogenesis inhibitor is osteoprotegerin.

10. The implant device of claim 2, wherein said osteoclastogenesis inhibitor is a bisphosphonate.

11. The implant device of claim 1, wherein the collagen is selected from the group consisting of human collagen type I, human collagen type II, human collagen type HI, human collagen type IV, human collagen type V, human collagen type VI, human collagen type VII, human collagen type VIII, human collagen type IX, human collagen type X, human collagen type XI, human collagen type XII, human collagen type XIII, human collagen type XIV, human collagen type XV, human collagen type XVI, human collagen type XVII, human collagen type XVII, human collagen type XIX, human collagen type XXI, human collagen type XXII, human collagen type XXIII, human collagen type XXIV, human collagen type XXV, human collagen type XXVI, human collagen type XXVII, and human collagen type XXVIII, and combinations thereof.

12. The implant device of claim 11, wherein the collagen is selected from the group consisting of hetero- or homo-trimers of human collagen type I, human collagen type II, human collagen type Ell, and mixtures or combinations thereof.

13. The implant device of claim 1, wherein the synthetic fibers are selected from the group consisting of polyglycolic acid, polydioxanone, polyglycolide, a copolymer containing glycolicacid units, a copolymer of methylmethacrylate and N-vinylpyyrolidone, polyamide, oxycellulose, copolymer of glycolic acid and trimethylene carbonate, polyesteramides, polylactide, polyetheretherketone, polymethylmethacrylate, fibrillated absorbable materials, and mixtures and combinations thereof.

14. The implant device of claim 1, wherein the composite implant device is comprised of a composite of collagen or synthetic fibers and a metal, a metal alloy, or a ceramic.

15. A method of making a composite spinal implant device comprising:
providing a composite implant composition comprising at least collagen and/or synthetic fibers;
forming the composite implant composition into a spinal implant; and
impregnating the collagen and/or synthetic fibers with a bioactive formulation.

16. The method of claim 15, wherein the collagen and/or synthetic fibers are impregnated prior to forming the composite implant composition into a spinal implant.

17. The method of claim 15, wherein the collagen and/or synthetic fibers are impregnated after forming the composite implant composition into a spinal implant.

18. The method of claim 15, further comprising processing the composite spinal implant after formation and impregnation with a procedure selected from the group consisting of sintering, heating, cooling, immersion in fluids or gases, surface treatments to roughen or make porous the surface of the implant, and sterilization.

19. A method of performing spinal surgery on a patient, comprising:
making an incision in a patient;
surgically accessing the area of the spine through the incision;
inserting the composite spinal implant device of claim 1; and
closing the incision.

20. A kit comprising the composite spinal implant device of claim 1.
